(19) United States
(12) Patent Application Publication
(10) Pub. No.: US 2010/0069493 A1
(43) Pub. Date: Mar. 18, 2010

(54) AQUEOUS PHARMACEUTICAL FORMULATION OF 4-[((4-CARBOXYBUTYL)-AMINO)METHYL]BENZOIC ACID

(75) Inventors: Bernd Kühn, Frankfurt (DE); Daniel Wagner, Köln (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER HEALTHCARE AG, Leverkusen (DE)

(21) Appl. No.: 12/307,452

(22) PCT Filed: Jun. 26, 2007
(86) PCT No.: PCT/EP2007/005625

§ 371 Date: Oct. 19, 2009

(30) Foreign Application Priority Data
Jul. 6, 2006 (DE) 10 2006 031 175.2

(51) Int. Cl. A61K 31/195

(52) U.S. Cl. 514/567

(57) ABSTRACT

The present invention relates to an aqueous pharmaceutical formulation which comprises 4-[((4-carboxybutyl)-{2-[(4-phenethylbenzyl)oxy]phenethyl}amino)methyl]benzoic acid or a salt thereof. The present invention relates in particular to an aqueous pharmaceutical formulation which comprises 4-[((4-carboxybutyl)-{2-[(4-phenethylbenzyl)oxy]phenethyl}amino)methyl]benzoic acid and 2-amino-2-(hydroxymethyl)-1,3-propanediol (trometamol).

The present invention relates to an aqueous pharmaceutical formulation which comprises 4-[(( 4-carboxybutyl)-{2-[(4-phenethylbenzyl)oxy]phenethyl}amino)methyl]benzoic acid or a salt thereof. The present invention relates in particular to an aqueous pharmaceutical formulation which comprises 4-[((4-carboxybutyl)-{2-[(4-phenethylbenzyl)oxy]phenethyl}amino)methyl]benzoic acid and 2-amino-2-(hydroxymethyl)-1,3-propanediol (trometamol).

4-[((4-Carboxybutyl)-{2-[(4-phenethylbenzyl)oxy]phenethyl}amino)methyl]benzoic acid (compound 1) is an activator of soluble guanylate cyclase having an effect on the cardiovascular system and corresponds to the following formula:

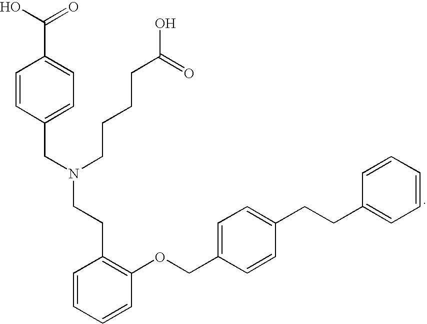

Compound 1 and its effect on soluble guanylate cyclase were described for the first time in WO 01/19780. However, WO 01/19780 does not describe any pharmaceutical preparations suitable for parenteral administration. There is a need, especially for the treatment of patients in intensive care units who are not capable of oral intake, for such an infusion solution which can be administered parenterally.

There are in particular two reasons in favour of parenteral use:
1) Oral use of the substance is possible, because of the short half-life of the substance, only with intake several times a day. At the same time, development of a conventional sustained release formulation is impeded by the fact that the substance is absorbed in sufficient quantities only from the upper part of the gastrointestinal tract.
2) Compound 1 is developed for use for acute heart failure. In this case, the active ingredient is normally administered in the intensive care setting. Oral administration of the drug is difficult in corresponding intensive care patients. Administration of an i.v. infusion by contrast ensures compliance and makes it possible for the dosage to be accurate and individually adapted. An i.v. solution is therefore the formulation of choice in this case.

It has been found during development work that the solubility in customary physiological solvents is insufficient to obtain physically stable solutions.

The object is, despite this problem, to formulate tolerated and, at the same time, storage-stable infusion solutions. The use of excipients in this connection is restricted to substances which are suitable for world-wide authorization of a parenteral medicament.

It has been found that adjusting the pH with customary buffers does not lead to a stable formulation with sufficient solubility.

Surprisingly, compound 1 forms with 2-amino-2-(hydroxymethyl)-1,3-propanediol (trometamol) a salt which has a sufficient solubility at a pH below 9.0 and thus can serve as basis for a stable and physiologically tolerated formulation. Trometanol is known to the skilled person. At the same time, the use of trometamol has the advantage that a fall in the pH during storage, and a precipitation of the active ingredient associated therewith, because of the reduced solubility, is prevented by the good buffering action of trometamol in the pH range between 8-9.

The reasonable daily dose for compound 1 is in the range from 2 to 20 mg, in a 50 ml bottle. These reasonable specifications for the dose of active ingredient (2-20 mg in 50 ml) result in a target active ingredient concentration in the solution in the range between 0.04 and 0.4 mg/ml. The solubility of the active ingredient in a suitable vehicle at room temperature ought to be at least a factor of 3 higher (safety margin), in particular also in order to avoid precipitations from the solutions during cold storage because the solubility is too low.

Because compound 1 has a plurality of ionizable groups (pKₐ₁: 4.0/pKₐ₂: 4.7/pKₐ₃: 8.3), experiments were carried out to improve the solubility by adjusting the pH to values greater than pH 7.4. However, there are limits to increasing the pH due to the use as infusion. Values of pH above pH 9.0 are borderline in relation to physiological tolerability. The use of customary buffers and bases did not lead to a usable formulation. Table 1 shows the solubilities of compound 1 in various buffer media.

TABLE 1
Solubility of compound 1 in various buffer media

| | | Saturation |
|---|---|---|
| | Actual | solubility |
| Name of the buffer | pH | [mg/ml] |
| Phosphate buffer solution | 6.0 | 0.0002 |
| Tetrabutylammonium buffer solution | 7.0 | 0.0128 |
| Phosphate buffer solution | 7.2 | 0.0084 |
| Phosphate buffer solution | 7.4 | 0.0215 |
| Saline-phosphate buffer | 7.4 | 0.0190 |
| Citric acid-phosphate buffer | 7.4 | 0.0131 |
| Phosphate buffer solution (0.2 M) | 7.5 | 0.0158 |
| Phosphate buffer solution (0.3 M) | 7.5 | 0.0013 |
| HEPES buffer solution | 7.5 | 0.0358 |
| Sodium citrate buffer solution | 7.8 | 0.1601 |
| Phosphate buffer solution | 8.0 | 0.0032 |
| Citric acid-phosphate buffer | 8.0 | 0.0642 |
| Phosphate buffer solution | 9.0 | 0.6826 |
| Ammonium chloride buffer solution | 9.5 | 75.7 |
| Diethanolamine buffer solution | 10.0 | 89.8 |

Trometamol can in principle be employed in the formulation in a concentration of from 0.002 to 0.2 M. A concentration of 0.01 M has proved to be particularly beneficial. If the concentrations are too low, the buffering action is insufficient to ensure a stable formulation. If the concentrations are too high, there must be a greater expectation of unwanted pharmacological effects, because the buffer burden on the physiological blood buffer is too great. Trometamol is employed intravenously in relatively high concentrations as active substance to counter hyperacidity. In this connection, the manufacturer (Braun, Melsungen) recommends that a concentration of 0.3 M not be exceeded in infusions. Thus, from the pharmacological/toxicological viewpoint, the aim should be a Trometamol concentration which is as low as possible.

A concentration of 0.5 mg/ml of compound 1 corresponds to a molarity of 0.001 M, so that complete salt formation for concentrations of up to 0.5 mg/ml is ensured by Trometamol concentrations of 0.0015 M and above.

Whereas the use of trometamol in the solutions is essential for the solubility of compound 1 at concentrations in the range from about 0.2 to 0.5 mg/ml, it is also advantageous to employ trometamol as excipient for infusion solutions even with solutions of compound 1 of lower concentration.

Dilution experiments on solutions with an active ingredient content of 0.05 mg/ml have shown that these can be diluted in a pH-stable and thus problem-free manner over a wide range only if they are buffered with a sufficient quantity of trometamol.

Without use of trometamol and on use of trometamol in a concentration of only 0.001 M, the pH falls greatly even with slight dilution (1+5). There is a high risk in this case that the active ingredient will precipitate on dilution with other infusion solutions (Table 2 a-c).

TABLE 2
Dilutability of solutions comprising compound 1

| Mixing ratio | | |
|---|---|---|
| 1 + 5 | 1 + 10 | 1 + 250 |

a) Without trometamol:

| | | | |
|---|---|---|---|
| Dilution medium | NaCl soln | NaCl soln | NaCl soln |
| Final volume after dilution | 251 ml | 275 ml | 300 ml |
| pH before dilution | 8.51 | 8.51 | 8.51 |
| pH of the dilution medium | 6.18 | 6.21 | 6.14 |
| pH after dilution | 6.40 | 6.58 | 6.70 |

b) With trometamol 0.001 molar:

| | | | |
|---|---|---|---|
| Dilution medium | NaCl soln | NaCl soln | NaCl soln |
| Final volume after dilution | 251 ml | 275 ml | 300 ml |
| pH before dilution | 8.69 | 8.69 | 8.69 |
| pH of the dilution medium | 6.25 | 6.06 | 6.12 |
| pH after dilution | 8.43 | 8.05 | 6.61 |

c) With trometamol 0.01 molar:

| | | | |
|---|---|---|---|
| Dilution medium | NaCl soln | NaCl soln | NaCl soln |
| Final volume after dilution | 251 ml | 275 ml | 300 ml |
| pH before dilution | 8.74 | 8.74 | 8.74 |
| pH of the dilution medium | 6.06 | 6.01 | 5.95 |
| Appearance of the solution | clear | clear | clear |

It was possible to show in titration experiments that an acid burden of 0.01 N hydrochloric acid in solutions without trometamol leads to such a large fall in pH even on addition of less than 1 ml to the total content of a 50 ml infusion bottle that the active ingredient precipitates. On addition of 0.001 M trometamol buffer, precipitations are to be observed on addition of about 5 ml of 0.01 N hydrochloric acid. A solution with a trometamol content of 0.01 M is by contrast stable to precipitations on addition of acid up to more than 35 ml of 0.01 N hydrochloric acid.

Solutions comprising compound 1 in a concentration of 0.05 mg/ml and trometamol in a concentration of 0.01 M can be diluted problem-free with standard infusion solutions over a wide range. The dilution experiments shown in Table 3 were carried out concerning this:

TABLE 3
Dilutability of solutions comprising compound 1
a) Dilutions with sodium chloride solution:

| | Mixing ratio | | | | | |
|---|---|---|---|---|---|---|
| | 1:2 | 1:4 | 1:10 | 1:20 | 1:40 | 1:200 |
| Dilution medium | NaCl | NaCl | NaCl | NaCl | NaCl | NaCl |
| | soln | soln | soln | soln | soln | soln |
| Final volume after | 10 ml | 20 ml | 50 ml | 100 ml | 200 ml | 1000 ml |
| dilution | | | | | | |
| pH after dilution | 8.67 | 8.65 | 8.57 | 8.54 | 8.40 | 7.85 |

b) Dilutions with other media:

| | Mixing ratio | | |
|---|---|---|---|
| | 1:10 | 1:10 | 1:10 |
| Dilution medium | Glucose soln | Water | Placebo soln |
| Final volume after dilution | 50 ml | 50 ml | 50 ml |
| pH after dilution | 8.37 | 8.61 | 8.72 |

All the dilution experiments were carried out in duplicate. No precipitations occurred with any of the solutions investigated. It emerged that there is a marked decrease in the pH values to the region below 8.0 only with dilutions greater than 1:40. Precipitation of the active ingredient is no longer a worry with such great dilutions, because the solubility limit of compound 1 is no longer reached owing to the large amount of dilution medium.

The invention makes it possible to prepare stable and tolerated infusion solutions comprising compound 1. The infusion solutions can be administered undiluted or diluted, e.g. as bypass infusion with other standard infusion solutions such as isotonic sodium chloride or glucose solution.

Dilute administration is advantageous in that the latitude for adaptations of concentration and dosage for optimal pharmacotherapy individually tailored for the patient is relatively large.

The invention thus relates to aqueous pharmaceutical formulations which comprise 4-[((4-carboxy-butyl)-{2-[(4-phenethylbenzyl)oxy]phenethyl}amino)methyl]benzoic acid (compound 1) or a salt thereof. The present invention relates in particular to an aqueous pharmaceutical formulation which comprises 4-[((4-carboxybutyl)-{2-[(4-phenethylbenzyl)oxy]phenethyl}amino)methyl]benzoic acid (compound 1) or a salt thereof and 2-amino-2-(hydroxymethyl)-1,3-propanediol (trometamol).

Physiologically acceptable salts may be metal or ammonium salts of the compound (1). Particularly preferred examples are sodium, potassium, magnesium or calcium salts, and ammonium salts derived from ammonia or organic amines such as, for example, ethylamine, di- or triethylamine, di- or triethanolamine, dicyclohexylamine, dimethylaminoethanol, arginine, lysine or ethylenediamine.

An aqueous pharmaceutical formulation in the context of the present invention is a formulation which substantially comprises water as solvent. However, depending on the infusion volume to be administered, it may comprise where appropriate water-miscible organic solvents in a proportion of up to 50% (M/V), preferably less than 30% (M/V), as long as they do not lead to an impairment of the physiological tolerability of the formulation. It is particularly preferred for the aqueous pharmaceutical formulation of the invention to comprise substantially no organic solvents.

The aqueous pharmaceutical formulation of the invention expediently comprises 0.0005% (M/V) (0.0001% M/V means 0.001 g/100 ml) to 1% (M/V), preferably 0.0025 to 0.25% (M/V), particularly preferably 0.005 to 0.025% (M/V) of the compound (1) or salts thereof These amounts by weight relate to the total volume of the formulation.

The amount of the tonicity agent used according to the invention is expediently chosen so that preparations having a tonicity of up to 430 mOsmol/kg, preferably 250 to 330 mOsmol/kg, are obtained.

The aqueous pharmaceutical formulation of the invention is expediently used for parenteral administration. Parenteral administration includes for example intravenous, intraarterial, subcutaneous, intramuscular and intraperitoneal administration, with intravenous administration having the greatest importance. As dose, 24 μg to 24 mg of active ingredient are regarded as expedient for intravenous infusion 1× a day. The infusion volume administered daily should not exceed 200 ml.

The aqueous pharmaceutical formulation of the invention may in addition to the ingredients used according to the invention comprise further excipients customary in the field of parenteral administration forms, such as, for example, acids and bases to adjust the pH, and customary preservatives, solubilizers and antioxidants.

The invention makes it possible to prepare stable and tolerated infusion solutions with the active ingredient of the compound (1). Easily handled solutions ready for infusion can be formulated. The solutions can be provided both in the form of glass infusion bottles or ampoules and in the form of flexible infusion bags or blown bottelpack® packs etc.

### EXAMPLES

### Example 1

### 0.5 mg/ml Infusion Solution

A 50 l bulk batch comprises:

| Composition | Function | Mass (g) |
|---|---|---|
| Active ingredient | | |
| Compound 1 | Active ingredient | 27.0000 |
| Excipients | | |
| Hydrochloric acid 1 M* | pH adjustment | 111.2016 |
| Sodium chloride | tonicity adjustment | 486.0000 |
| Sodium hydroxide solution 0.1 M* | pH adjustment | 675.0000 |
| Trometamol | solubilization, | 65.4156 |
| | pH stabilization | |
| Water for injections | solvent | 52889.18 |
| Total amount for bottling | | 54253.80 |
| (equivalent to 50000 ml) | | |

The active ingredient is dissolved in a glass or steel container in the stated amount of sodium hydroxide solution.

Then, trometamol, hydrochloric acid and 5% of the total amount of water are mixed and stirred until the trometamol has dissolved. The pH of this solution should be between 8.6 and 8.8.

In parallel with this, the sodium chloride is dissolved in 90% of the amount of water. The solution is adjusted to pH 8.0-8.5 with sodium hydroxide solution. The amount of 0.1 N NaOH required for this is recorded.

Finally, while stirring, firstly the trometamol solution and then the active ingredient concentrate are added. The pH of the solution is checked and adjusted to pH 8.8 with 1 M hydrochloric acid or 0.1 M sodium hydroxide solution. Stirring is continued until a clear solution results.

The finished solution is sterilized by filtration through a filter of pore width 0.22 μm (Pall Ultipor Nylon 66) under a gauge pressure of 1.6 bar of nitrogen, bottled in 50 ml glass infusion bottles and closed with PTFE-laminated chlorobutyl rubber infusion stoppers and aluminium crimped caps. The bottled solutions containing compound (1) are autoclaved at 121° C. for at least 20 minutes.

### Example 2

### 0.05 mg/ml Infusion Solution

A 50 ml infusion bottle contains:

| Composition | Function | Mass (g) |
|---|---|---|
| Active ingredient | | |
| Compound (1) | Active ingredient | 0.0025 |
| Excipients | | |
| Hydrochloric acid 1 M | pH adjustment | 0.103-0.150 |
| Sodium chloride | tonicity adjustment | 0.450 |
| Sodium hydroxide | pH adjustment | 0.063-0.150 |
| solution 0.1 M | | |
| Trometamol | solubilization, | 0.061 |
| | pH stabilization | |
| Water for injections | solvent | 49.4165-49.5505 |
| Total amount for bottling | 50.0 ml = | 50.23 |
| Overage | 1.0 ml | |
| (in order to ensure | | |
| removal of 50 ml) | | |
| Total amount in bottle | 51.0 ml = | 51.23 |

Preparation takes place in analogy to Example 1.

### Example 3

### 0.125 mg/ml Infusion Solution

One litre of bulk solution comprises:

| Composition | Function | Mass (g) | |
|---|---|---|---|
| Active ingredient | | | |
| Compound (1) | Active ingredient | 0.1250 | |
| Excipients | | | |
| Hydrochloric acid 1 M* | pH adjustment | 2.2666 | |
| Sodium chloride | tonicity adjustment | 9.0000 | |
| Sodium hydroxide solution 0.1 M* | pH adjustment | 3.1250 | |
| Trometamol | solubilization, | 1.3333 | |
| | pH stabilization | | |
| Water for injections | solvent | 988.8474 | |
| Total amount for bottling | | 1004.70 | g |
| (equivalent to 1000 ml) | | | |

*to adjust the pH to pH 8.8. Amount may vary.

Preparation takes place in analogy to Example 1.

1. An aqueous pharmaceutical formulation comprising 4-[((4-carboxybutyl)-{2-[(4-phenethylbenzyl)oxy]phenethyl}amino)methyl]benzoic acid (compound 1) or a salt thereof and 2-amino-2-(hydroxymethyl)-1,3-propanediol (trometamol).

2. The aqueous pharmaceutical formulation according to claim 1, wherein compound (1) or a salt thereof is present in an amount of 0.0005 to 1% (based on the amount of compound 1).

3. The aqueous pharmaceutical formulation according to claim 1, further comprising a tonicity agent in an amount such that formulation has a tonicity of 250 to 430 mOsmol/kg.

4. The aqueous pharmaceutical formulation according to claim 1, wherein the mmol/l 2-amino-2-(hydroxymethyl)-1,3-propanediol (trometamol) is from 0.1 to 300 mmol/l.

5. The aqueous pharmaceutical formulation according to claim 1 for parenteral administration in humans and animals.

6. (canceled)

7. (canceled)

8. A method of preparing an aqueous formulation of the compound (1) or of a salt thereof, in which a solution of the compound (1) or of a salt thereof having a concentration of the compound (1) or of a salt thereof of more than 0.0005% (M/V) up to the saturation concentration of compound 1 or of the salt thereof at room temperature is brought with an infusion vehicle solution comprising 2-amino-2-(hydroxymethyl)-1,3-propanediol (trometamol) to a use concentration suitable for parenteral administration.

9. The method of according to claim 8, in which the solution comprises compound (1) at a concentration of more than 0.0005% (M/V) up to 1% (M/V) (based on the amount of compound 1) is used.

10. (canceled)
